(19) United States
(12) Patent Application Publication
(10) Pub. No.: US 2023/0372303 A1
(43) Pub. Date: Nov. 23, 2023

(54) APPLICATIONS OF ZWITTERIONS IN OPHTHALMIC TOPICAL CREAM COMPOSITIONS AND PREPARATIONS

(71) Applicant: GLAUKOS CORPORATION, Aliso Viejo, CA (US)

(72) Inventors: Xiaojun Michael Liu, Lake Forest, CA (US); James Jane-Guo Shiah, Irvine, CA (US); Gabriella Szekely, Irvine, CA (US)

(21) Appl. No.: 18/319,380

(22) Filed: May 17, 2023

Related U.S. Application Data
(60) Provisional application No. 63/364,914, filed on May 18, 2022.

(51) Int. Cl. A61K 31/4178; A61K 47/18; A61K 9/00

(52) U.S. Cl. CPC A61K 31/4178; A61K 47/183; A61K 9/0014; A61K 9/0048

(57) ABSTRACT

An ophthalmic topical cream composition includes a topical cream comprising at least one zwitterion; and an active pharmaceutical ingredient (API) dispersed in the topical cream. The ophthalmic topical cream composition and API are formulated for topical application and treatment of various disorders such as dry eye, presbyopia, intraocular pressure and blepharitis.

### INCORPORATION BY REFERENCE TO ANY PRIORITY APPLICATIONS

This application claims priority to U.S. Provisional Patent Application No. 63/364,914, entitled “NOVEL APPLICATIONS OF ZWITTERIONS IN OPHTHALMIC TOPICAL CREAM COMPOSITIONS AND PREPARATIONS,” filed on May 18, 2022, which is hereby incorporated by reference in its entirety and for all purposes.

### BACKGROUND

### Field

This disclosure relates to ophthalmic topical cream composition and methods of using them to treat disorders and diseases of the eye, particularly by administration to an outer surface of an eyelid of the patient's eye.

### Description of the Related Art

Eye drops are the typical way of administrating ophthalmic medicine to patients. For example, U.S. Pat. No. 10,610,518 discloses pilocarpine formulations and methods of administering them as eye drops. This route is simple and non-invasive but achieves only limited control over patient's compliance, dose regimen, and duration. Eye drops may lead to undesirable efficacy and clinical results if subjects use them incorrectly. See Souto, E. B., et al., “Advanced Formulation Approaches for Ocular Drug Delivery: State-Of-The-Art and Recent Patents”, Pharmaceutics. 2019, 11, 460: 1-29. Other ophthalmic formulations have been developed that are said to be suitable for administration by topical application to an outer surface of an eyelid of a subject. See U.S. Pat. No. 9,034,830. However, there remains a need for improved ophthalmic treatment compositions and methods. For example, it is extremely challenging to maintain both physical and chemical stability of an active pharmaceutical ingredient (API) in semi-solid dosage forms. One of the difficulties is that the selected thickener(s) used in the formulations may not be able to achieve and maintain stable cream textures desired for an ophthalmic topical cream. Another challenge is to achieve and maintain chemical stability of the active pharmaceutical ingredient (API) throughout the shelf life of the ophthalmic topical cream.

### SUMMARY

Various embodiments provide ophthalmic topical cream compositions. In an embodiment, an ophthalmic topical cream composition comprises a topical cream comprising at least one zwitterion and an active pharmaceutical ingredient (API) dispersed in the topical cream.

In some embodiments, the at least one zwitterion can be present in an amount effective to reduce degradation of the API and enhance viscosity of the topical cream. In some embodiments, the at least one zwitterion can have a concentration in the topical cream of about 5 mM to about 600 mM. In some embodiments, the at least one zwitterion can have a concentration in the topical cream of about 10 mM to about 500 mM. In some embodiments, the topical cream can have a viscosity of about 25,000 cPs to about 400,000 cPs. In some embodiments, the topical cream can have a viscosity of about 50,000 cPs to about 150,000 cPs.

In some embodiments, the at least one zwitterion can comprise at least one of an amino acid, a peptide, a protein, a phosphatidylcholine, and a betaine. In some embodiments, the topical cream can be an oil-in-water or water-in-oil emulsion system. In some embodiments, the at least one amino acid can comprise lysine, arginine, histidine, or a combination thereof. In some embodiments, the at least one amino acid can comprise lysine and arginine. In some embodiments, the lysine and arginine in the topical cream each independently can have a concentration of about 10 mM to about 500 mM. In some embodiments, the at least one amino acid can comprise histidine and arginine. In some embodiments, the histidine and arginine in the topical cream each independently can have a concentration of about 10 mM to about 500 mM. In some embodiments, the at least one amino acid can comprise histidine and lysine. In some embodiments, the histidine and lysine in the topical cream each independently can have a concentration of about 10 mM to about 500 mM.

In some embodiments, the at least one zwitterion can comprise a zwitterionic polymer or a polymeric zwitterion.

In some embodiments, the topical cream further can comprise a crosslinked acrylic acid polymer. In some embodiments, the topical cream can comprise less than about 1% (w/w) of a crosslinked acrylic acid polymer. In some embodiments, the topical cream can have a pH of about 3.0 to about 7.8. In some embodiments, the topical cream can comprise about 0.002% to about 10% (w/w) of the API. In some embodiments, the API can comprise pilocarpine, atropine, travoprost, physostigmine, donepezil, timolol, loteprednol, brimonidine, and/or a salt thereof. In some embodiments, the topical cream can comprise an amount of the API that is effective to alleviate the symptoms of dry eye, presbyopia, myopia, blepharitis, glaucoma, and/or edema.

In addition, various embodiments of a method for administering an ophthalmic topical cream composition to a patient's eye are provided. In an embodiment, a method of treating dry eye, presbyopia, glaucoma, and/or myopia comprises administering an effective amount of the ophthalmic topical cream composition to a subject in need thereof. In some embodiments, the ophthalmic topical cream composition is applied to an outer surface of an eyelid of the subject's eye.

### BRIEF DESCRIPTION OF THE DRAWINGS

FIG. 1 illustrates the stability of an ophthalmic topical cream composition having 10 mM Histidine as a function of storage time comparing to the stability of an ophthalmic topical cream composition in a non-buffered solution at room temperature and 40° C.

FIG. 2 illustrates the stability of an ophthalmic topical cream composition having 10 mM Arginine as a function of storage time comparing to the stability of an ophthalmic topical cream composition in a non-buffered solution at room temperature and 40° C.

FIG. 3 illustrates the stability of an ophthalmic topical cream composition having 5 mM Histidine and 5 mM Lysine as a function of storage time comparing to the stability of an ophthalmic topical cream composition in a non-buffered solution at room temperature and 40° C.

FIG. 4 illustrates the stability of an ophthalmic topical cream composition having 5 mM Arginine and 5 mM Lysine as a function of storage time comparing to the stability of an ophthalmic topical cream composition in a non-buffered solution at room temperature and 40° C.

FIG. 5 illustrates the stability of an ophthalmic topical cream composition having 5 mM Arginine and 5 mM Histidine as a function of storage time comparing to the stability of an ophthalmic topical cream composition in a non-buffered solution at room temperature and 40° C.

FIG. 6 illustrates the stability of an ophthalmic topical cream composition having 10 mM Lysine as a function of storage time comparing to the stability of an ophthalmic topical cream composition in a non-buffered solution at room temperature and 40° C.

FIG. 7 illustrates the stability of an ophthalmic topical cream composition having 25 mM Lysine as a function of storage time comparing to the stability of an ophthalmic topical cream composition in a non-buffered solution at room temperature and 40° C.

FIG. 8 illustrates the stability of an ophthalmic topical cream composition having 10 mM Histidine, 10 mM Arginine, or 10 mM Lysine as a function of storage time comparing to the stability of an ophthalmic topical cream composition in a non-buffered solution at 40° C.

FIG. 9 illustrates the stability of an ophthalmic topical cream composition having 10 mM Lysine or 25 mM Lysine as a function of storage time at 40° C. comparing to the stability of an ophthalmic topical cream composition in a non-buffered solution at 40° C.

FIG. 10 illustrates the stability of an ophthalmic topical cream composition having 5 mM Histidine and 5 mM Lysine, 5 mM Arginine and 5 mM Lysine, or 5 mM Arginine and 5 mM Histidine as a function of storage time at 40° C. comparing to the stability of an ophthalmic topical cream composition in a non-buffered solution at 40° C.

### DETAILED DESCRIPTION

Ophthalmic topical cream compositions have now been developed that contain a topical cream that includes at least one zwitterion and an active pharmaceutical ingredient (API) dispersed in the topical cream. In some embodiments, the at least one zwitterion may comprise at least one positive charged functional group and at least one negative charged functional group. In some embodiments, the at least one zwitterion may comprise both positive and negative electrical charges. In some embodiments, the net charge of the at least one zwitterion can be zero. In some embodiments, the net charge of the at least one zwitterion can be negative in neutral pH environment. For example, some amino acids, such as glutamic acid and aspartic acid, can be negatively charged in a neutral pH environment. In some embodiments, the net charge of the at least one zwitterion can be positive in a neutral pH environment. For example, some amino acids, such as lysine, arginine, and histidine, can be positively charged in a neutral pH environment. In various embodiments, this at least one zwitterion can impart surprisingly high levels of desirable features to the ophthalmic treatment compositions, such as improved chemical and physical stability of APIs, emollience, desirable and stable texture.

The ophthalmic topical cream compositions normally can contain active pharmaceutical ingredient(s) (APIs), thickening agents, stabilizing agents, tonicity agents and antimicrobial preservatives. In some embodiments, the ophthalmic topical cream can be an oil-in-water (o/w) emulsion system. This o/w emulsion system delivers active pharmaceutical ingredients (APIs) to the designated site, for example, topical skin, such as eye lid area.

It is observed that the processes to achieve a semi-solid texture for ophthalmic topical cream may be complex and challenging. Moreover, it is difficult to maintain both physical and chemical stability of the API in the semi-solid textures and matrices. One of the difficulties may be that the selected thickener(s) used in the ophthalmic topical cream compositions may not be able to achieve and maintain a stable and desirable cream textures for an ophthalmic topical cream over a long period of time, such as a shelf life of the product. Another difficulty may be to achieve and maintain chemical stability of API throughout the shelf life of the ophthalmic topical cream. Surprisingly, the presence of the at least one zwitterion in the ophthalmic topical cream compositions may help increase the viscosity of the ophthalmic topical cream to the level that the ophthalmic topical cream is semi-solid or cream textures or matrices. Moreover, the inclusion of the at least one zwitterion may help adjust the pH of the ophthalmic topical cream composition and help maintain the chemical and physical stability of the APIs.

### Ophthalmic Topical Cream Compositions

Various embodiments provide ophthalmic topical cream compositions, comprising:
a topical cream comprising at least one zwitterion; and
an active pharmaceutical ingredient (API) dispersed in the topical cream.

Various suitable zwitterions are commercially available and/or may be readily synthesized by those skilled in the art. In some embodiments, the at least one zwitterion presents in an amount effective to reduce degradation of the API and enhance viscosity of the topical cream. In some embodiments, the at least one zwitterion can have a concentration in the topical cream of about 5 mM to about 600 mM, about 10 mM to about 500 mM, about 30 mM to about 60 mM, about 80 mM to about 120 mM, about 5 mM to about 30 mM, or any other amount in a range defined by any two of the foregoing concentration values as endpoints. In some embodiments, the topical cream can have a viscosity of about 25,000 cPs to about 400,000 cPs, about 50,000 cPs to about 150,000 cPs, about 50,000 cPs to about 100,000 cPs, or any other amount in a range defined by any two of the foregoing viscosity values as endpoints.

In an embodiment, the at least one zwitterion contained in the ophthalmic topical cream composition may comprise at least one of an amino acid, a peptide, a protein, a phosphatidylcholine, and a betaine. In some embodiments, the at least one amino acid may comprise lysine, arginine, histidine, or a combination thereof. In some embodiments, the ophthalmic topical cream composition may comprise or may be lysine and arginine. In those embodiments, the lysine and arginine may each independently have a concentration of about 5 mM to about 500 mM, about 5 mM to about 100 mM, about 5 mM to about 50 mM, about 5 mM to about 30 mM, about 5 mM to about 15 mM, or any other amount in a range defined by any two of the foregoing concentration values as endpoints. In some embodiments, the ophthalmic topical cream composition may comprise or may be lysine and histidine. In those embodiments, the lysine and histidine may each independently have a concentration of about 5 mM to about 500 mM, about 5 mM to about 100 mM, about 5 mM to about 50 mM, about 5 mM to about 30 mM, about 5 mM to about 15 mM, or any other amount in a range defined by any two of the foregoing concentration values as endpoints. In some embodiments, the ophthalmic topical cream composition may comprise or may be arginine and histidine. In those embodiments, the arginine and histidine may each independently have a concentration of about 5 mM to about 500 mM, about 5 mM to about 100 mM, about 5 mM to about 50 mM, about 5 mM to about 30 mM, about 5 mM to about 15 mM, or any other amount in a range defined by any two of the foregoing concentration values as endpoints.

In another embodiment, the at least one zwitterion contained in the ophthalmic topical cream composition may comprise a zwitterionic polymer or a polymeric zwitterion.

In some embodiments, the topical cream can further comprise a crosslinked acrylic acid polymer. In some embodiments, the topical cream can comprise less than about 1% (w/w), about 0.5% (w/w), or about 0.2% (w/w) of a crosslinked acrylic acid polymer. In some embodiments, the topical cream can have a pH of about 3.0 to about 7.8, about 3.0 to about 7.0, about 3.0 to about 5.0, about 3.0 to about 4.0, about 3.5, about 3.7, or any other pH in a range defined by any two of the foregoing pH values as endpoints.

In some embodiments, the topical cream can comprise about 0.002% to about 10% (w/w) of the API. In some embodiments, the concentration of the API can be about 0.1% to about 10% (w/w), about 0.5% to about 8% (w/w), about 1% to about 8% (w/w), or any other amount in a range defined by any two of the foregoing concentration values as endpoints. In some embodiments, the API can comprise pilocarpine, atropine, travoprost, physostigmine, donepezil, timolol, loteprednol, brimonidine, and/or a salt (e.g., a pharmaceutically acceptable salt) of any of the foregoing. In some embodiments, the topical cream can comprise an amount of the API that is effective to alleviate the symptoms of dry eye, presbyopia, myopia, blepharitis, glaucoma, and/or edema.

In various embodiments, the ophthalmic topical cream composition is formulated to treat dry eye, presbyopia, glaucoma, and/or myopia. In some embodiments, the ophthalmic topical cream composition can be administered in an effective amount to a subject in need. In some embodiments, the ophthalmic topical cream composition can be applied to an outer surface of an eyelid of the subject's eye. After such application, the higher viscosity developed by the ophthalmic treatment composition after application on the eyelid allows it to remain in place, thereby providing the subject with additional time prior to being rubbed into the eye.

### Stability of the Ophthalmic Topical Cream Composition

As discussed above, surprisingly, the addition of zwitterion may control the pH of the ophthalmic topical cream composition, thus may help maintain the chemical and physical stability of the API. In some embodiments, the zwitterion(s) can be dissolved in an aqueous solution as a buffer agent to control the formulation pH. Advantageously, the use of zwitterion as a buffer agent may achieve synergistic effect in both physical and chemical stability in the cream-based matrix for controlled release of APIs over a desirable period. In some embodiments, the zwitterion may be an amino acid. In some embodiments, the at least one zwitterion can include at least one of an amino acid, a peptide, a protein, a phosphatidylcholine, and a betaine. In some embodiments, the at least one zwitterion can be at least one of an amino acid, a peptide, a protein, a phosphatidylcholine, and a betaine. In some embodiments, the amino acid can include lysine, arginine, histidine, or a combination thereof. In some embodiments, the amino acid can be lysine, arginine, histidine, or a combination thereof. In some embodiments, the at least one amino acid can comprise lysine and arginine. In some embodiments, the at least one amino acid can be lysine and arginine. In some embodiments, the at least one amino acid can comprise lysine and histidine. In some embodiments, the at least one amino acid can be lysine and histidine. In some embodiments, the at least one amino acid can comprise arginine and histidine. In some embodiments, the at least one amino acid can be arginine and histidine.

Non-limiting examples of suitable amounts of zwitterion and API for ophthalmic topical cream compositions are illustrated and summarized in Table 1 with pilocarpine as the exemplified drug (API).

TABLE 1

| | Buffer | Pilocarpine | Preservative | | |
|---|---|---|---|---|---|
| No. | Strength | (wt. %) | (wt. %) | pH | e-Beam |
| F27 | Non-buffered | 8 | 0.2% Paraben | 3.5 | None |
| F105 | 10 mM Histidine | 8 | 0.2% Paraben | 3.5 | 25 kGy |
| F106 | 10 mM Arginine | 8 | 0.2% Paraben | 3.5 | 25 kGy |
| F107 | 5 mM Lysine + | 8 | 0.2% Paraben | 3.5 | 25 kGy |
| | 5 mM Histidine | | | | |
| F108 | 5 mM Lysine + | 8 | 0.2% Paraben | 3.5 | 25 kGy |
| | 5 mM Arginine | | | | |
| F109 | 5 mM Histidine + | 8 | 0.2% Paraben | 3.5 | 25 kGy |
| | 5 mM Arginine | | | | |
| F121 | 10 mM Lysine | 8 | 0.06% Paraben | 3.5 | 25 kGy |
| F122 | 25 mM Lysine | 8 | 0.06% Paraben | 3.5 | 25 kGy |

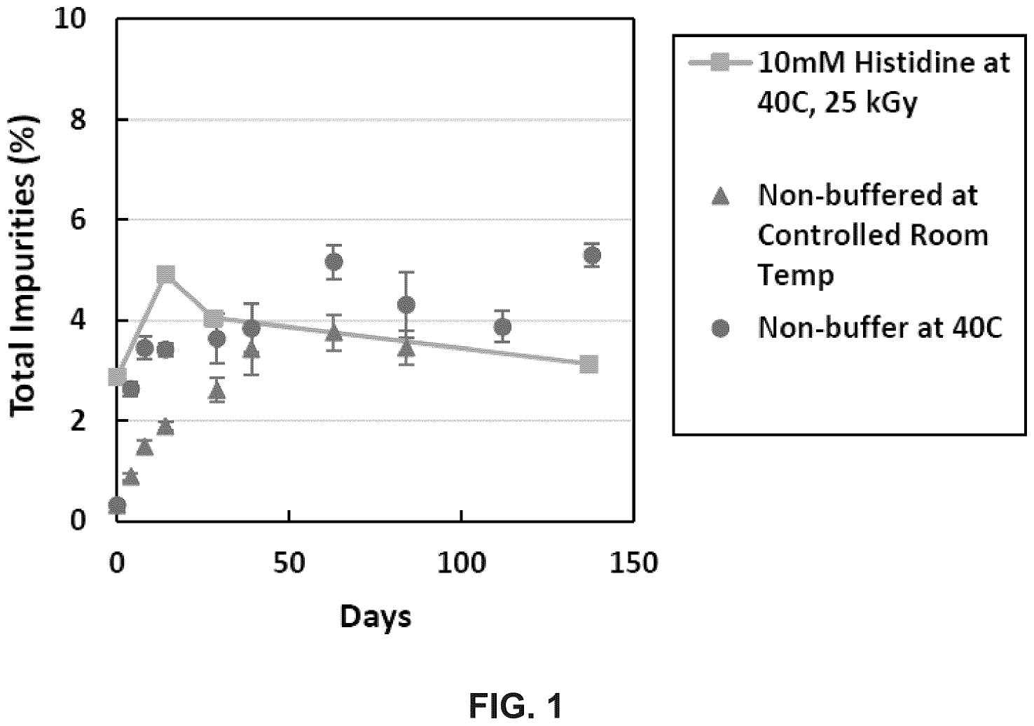

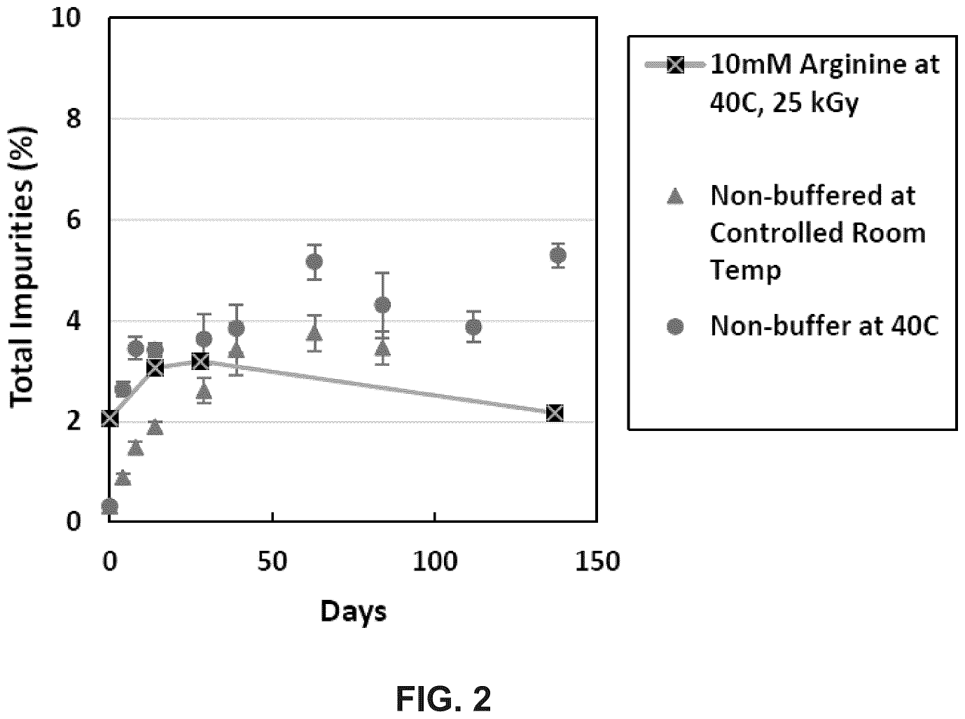

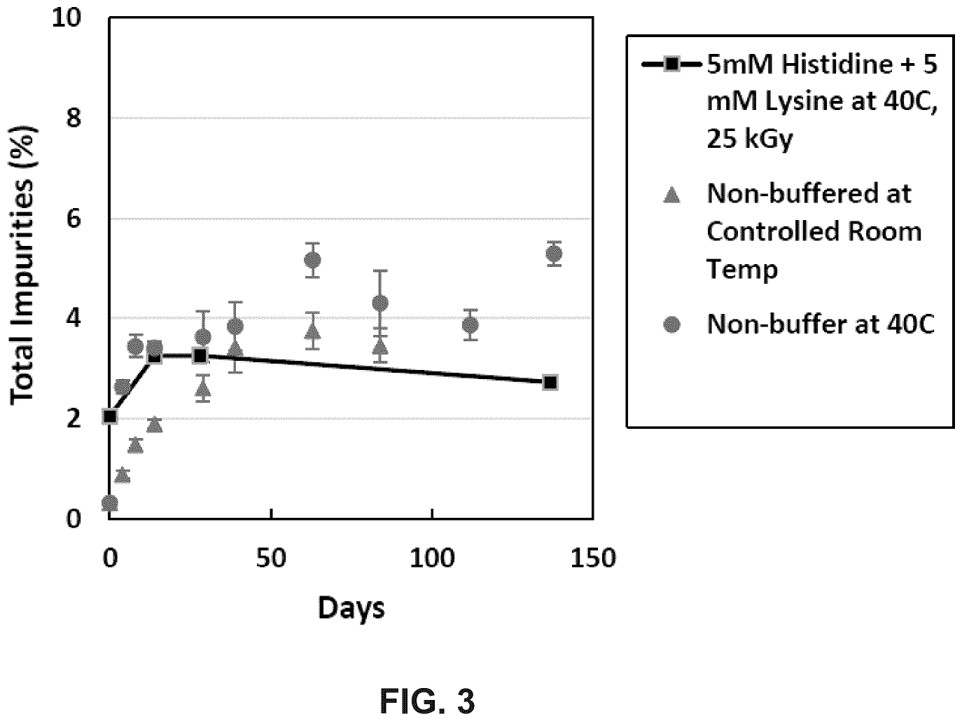

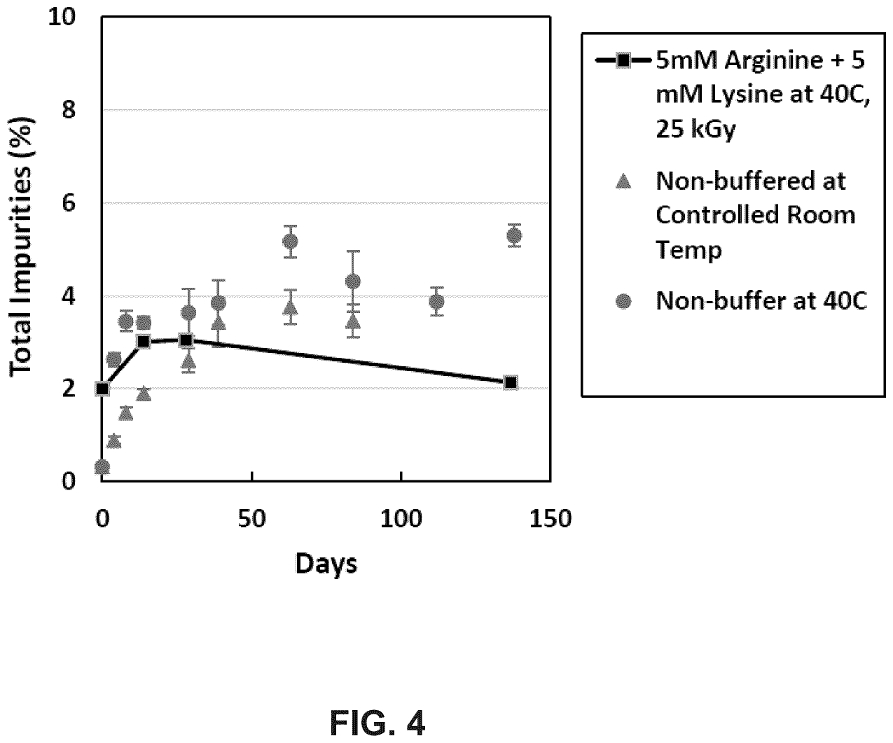

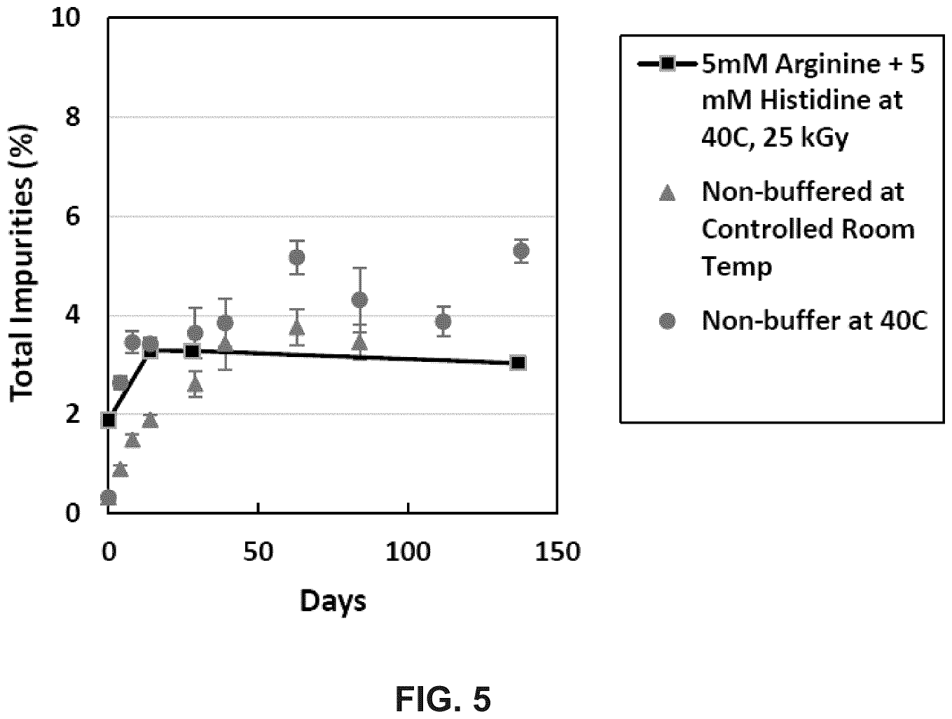

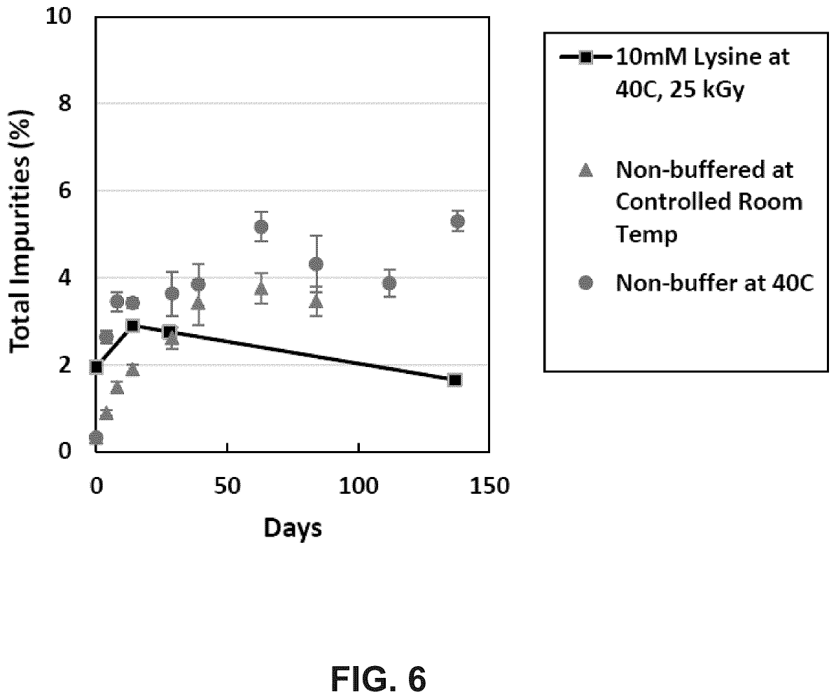

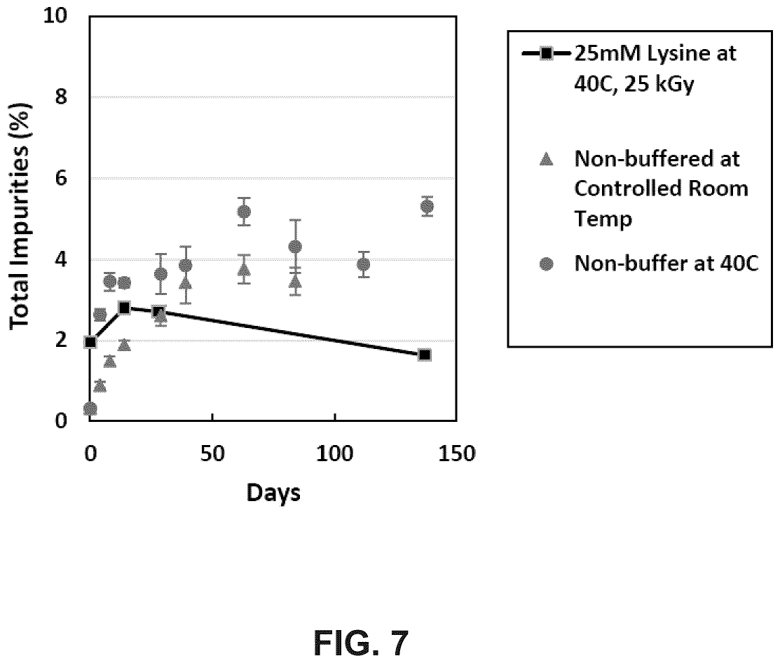

As shown in Table 1, these non-limiting examples of ophthalmic topical cream compositions were formulated and then sterilized by e-Beam sterilization at dose of 25 kGy, except that the composition F27 was not sterilized by e-Beam. As illustrated in Table 1, the pH of the various ophthalmic topical cream compositions was maintained at about 3.5. F27 is a control formulated with a non-buffered solution to be compared with the ophthalmic topical cream compositions comprising at least one zwitterion as a buffer agent to control the formulation pH. F105 (FIG. 1) contains10 mM Histidine as a buffer agent, F106 (FIG. 2) contains 10 mM Arginine, F107 (FIG. 3) contains 5 mM Lysine and 5 mM Histidine, F108 (FIG. 4) contains 5 mM Lysine and 5 mM Arginine, F109 (FIG. 5) contains 5 mM Histidine and 5 mM Arginine, F121 (FIG. 6) contains 10 mM Lysine, and F122 (FIG. 7) contains 25 mM Lysine. All the composition examples in Table 1 were formulated with the same concentrations of thickeners and exemplified drug pilocarpine using the same process, except that the composition F27 was not sterilized by e-Beam.

FIGS. 1-7 illustrate the stability of various ophthalmic topical cream compositions comprising at least one functional amino acid in the buffer solution as a function of storage time as compared to the stability of an ophthalmic topical cream composition formulated with a non-buffered solution at room temperature and 40° C. Room temperature is a temperature in the range of about 15° C. to about 25° C. In some embodiments, room temperature is 25° C. The impurities measured are degradation products of the API, which is pilocarpine in the study described here.

As shown in FIGS. 1-7, the total impurities from the composition F27 (non-buffered solution) continued to grow over a period of four months. In addition, the amounts of impurities from the composition F27 at a higher temperature (40° C.) is more than the amounts of impurities at a controlled room temperature. As illustrated in FIGS. 1-7, the amounts of impurities from the composition F27 at 40° C. is above 5% at the end of testing period. In contrast, the amounts of impurities from compositions with one functional amino acid at a storage temperature of 40° C. are all less than the impurities from the composition without any amino acids in the buffer at a storage temperature of 40° C. and room temperature at the end of testing period. As illustrated by FIGS. 1-7, surprisingly, the amounts of impurities from compositions with one amino acid at a storage temperature of 40° C. are controlled at about 3% or less.

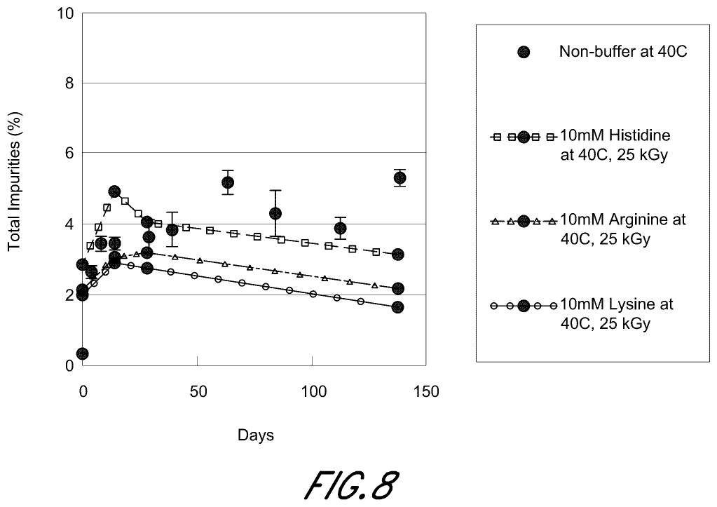

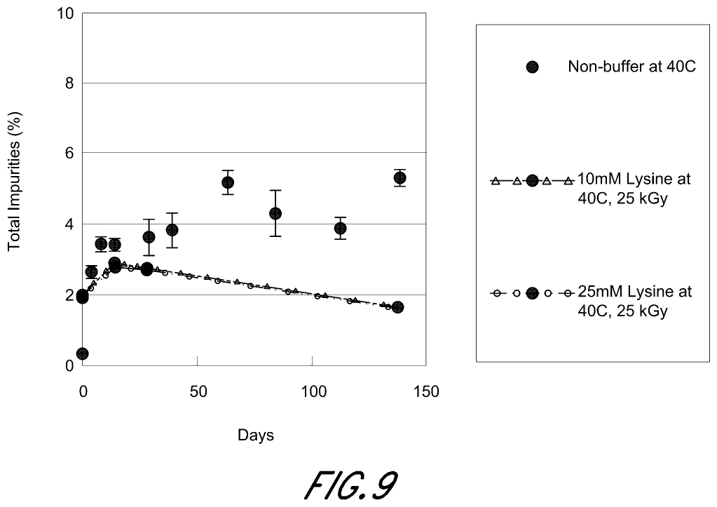

As illustrated in FIGS. 8 and 9, among all the composition examples comprising one functional amino acid, the compositions comprising 10 mM or 25 mM of Lysine showed the lowest amount of total impurities. As shown in FIG. 9, it is noticed that the compositions comprising 10 mM or 25 mM of Lysine had similar amounts of total impurities when measured at different stability time points of the test.

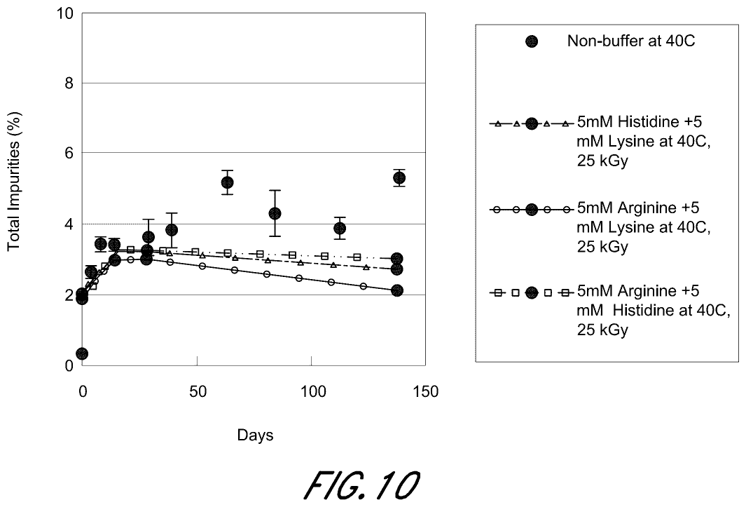

FIG. 10 illustrates the amounts of impurities from the composition examples comprising two functional amino acids. As illustrated in FIG. 10, the exemplified drug pilocarpine is the most stable in the composition comprising 5 mM Lysine and 5 mM Arginine comparing to the other two composition examples. Surprisingly, FIG. 10 illustrates that the combinations of two different amino acids have synergistic effect in stabilizing the pilocarpine. Among the various examples, the combination of 5 mM of arginine and 5 mM of lysine shows the most significant effect on pilocarpine stabilization at the accelerated stability condition.

The results as illustrated in FIGS. 1-10 show that the inclusion of the at least one amino acid helps maintain the chemical stability of the API. Moreover, it is noted that among the various examples of functional amino acids used in the compositions, lysine may be the most effective stabilizing agent for the exemplified drug pilocarpine, followed by arginine and histidine. In addition, among the compositions comprising two functional amino acids, the combination of lysine and arginine, as well as the combination of lysine and histidine show strong chemical protection for the pilocarpine over the four-month testing period at accelerated storage conditions.

### Texture of the Ophthalmic Topical Cream Composition

As mentioned above, in some embodiments, the ophthalmic treatment composition comprising at least one zwitterion may comprise an oil-in-water emulsion system in which the oil phase is dispersed into the aqueous phase. It is noted that maintaining the semi-solid or cream body matrix of such an oil-in-water emulsion system is important to achieve desirable physical stability. Therefore, it is important to select thickener(s) with appropriate concentrations, polymer molecular weight and polydispersity. In addition, the selection of appropriate medium and/or ingredient synergies may play an important role in the product development process. To achieve a semi-solid and/or cream texture for an ophthalmic topical cream composition, it is noted that the viscosity of the emulsion system may be about 50,000 cPs to 150,000 cPs.

Various examples of ophthalmic treatment composition are illustrated in Table 2 to show the importance of appropriate media for ophthalmic treatment composition. In all the examples, the drug exemplified was atropine sulfate, 4% of Carbomer was used as a thickener, and the pH of the formulations was adjusted to approximately 3.7 in all the examples.

TABLE 2

| | Atropine | Carbomer | Aqueous | | Viscosity | |
|---|---|---|---|---|---|---|
| No. | (wt. %) | (wt. %) | Media | pH | (cPs) | Notes |
| AS2R | 1 | 4 | Water | 3.7 | <3,000 | Phase separation |
| | | | | | | detected |
| AS9 | 1 | 4 | 500 mM | 3.7 | 85,000 | No phase |
| | | | citrate | | | separation for |
| | | | buffer | | | 1 month after |
| | | | | | | forced |
| | | | | | | centrifugation |
| AS12 | 1 | 4 | 200 mM | 3.7 | 70,000 | Minimal phase |
| | | | citrate | | | separation for |
| | | | buffer | | | 1 month after |
| | | | | | | forced |
| | | | | | | centrifugation |
| AS13 | 1 | 4 | 200 mM | 3.7 | 68,000 | Minimal phase |
| | | | potassium | | | separation for |
| | | | citrate | | | 1 month after |
| | | | | | | forced |
| | | | | | | centrifugation |
| AS14 | 1 | 4 | 100 mM | 3.7 | 362,000 | No phase |
| | | | Lysine | | | separation for |
| | | | | | | 1 month after |
| | | | | | | forced |
| | | | | | | centrifugation |
| AS15 | 1 | 4 | 100 mM | 3.7 | 298,500 | No phase |
| | | | Histidine | | | separation for |
| | | | | | | 1 month after |
| | | | | | | forced |
| | | | | | | centrifugation |
| AS17 | 1 | 4 | 10 mM | 3.7 | <3,000 | Phase separation |
| | | | Lysine | | | detected |
| AS20 | 1 | 4 | 50 mM | 3.7 | 71,130 | Minimal phase |
| | | | Lysine | | | separation |
| | | | | | | detected at |
| | | | | | | the initial |
| | | | | | | after forced |
| | | | | | | centrifugation |
| AS21 | 1 | 4 | 25 mM | 3.7 | <3,000 | Phase separation |
| | | | Lysine | | | detected |
| AS24 | 1 | 4 | 25 mM | 3.7 | <3,000 | Phase separation |
| | | | Histidine | | | detected |

As shown in Table 2, water (AS2R) may be an appropriate medium for forming a lotion, but may not be an appropriate medium to form a cream texture as the viscosity is lower than 3000 cPs. In contrast, as shown in Table 2, various examples of buffers or amino acid solutions can be more appropriate medium to form a cream texture as the viscosities are higher than about 50,000 cPs and below about 150,000 cPs. In addition, as shown in Table 2, it is noted that the medium containing amino acid buffers may be stronger viscosity enhancers than non-amino acid buffers to form the desired cream texture for ophthalmic treatment compositions. To achieve a viscosity of about 70,000 cPs, a citrate or potassium buffer with a concentration of about 200 mM may be needed. In comparison, a solution with a concentration of only 50 mM Lysine achieved a viscosity of about 71,130 cPs. When the concentration of Lysine was increased to about 100 mM, the viscosity was significantly increased to about 362,000 cPs (AS14). In some embodiments, a concentration of about 50 mM Lysine can offer a desirable viscosity, such that ophthalmic treatment composition may have a cream texture, with no phase separation observed.

As illustrated by the results in the Table 2, the inclusion of the at least one zwitterion may help enhance the viscosity of the ophthalmic topical cream composition and make the texture of the ophthalmic topical cream composition semi-solid or cream-like. In some embodiments, the inclusion of the at least one zwitterion may help maintain the physical stability of the ophthalmic topical cream composition and no phase separation is observed.

Methods of Treatment with Ophthalmic Topical Cream Composition

Various embodiments provide methods of treating a disease or disorder of the eye by topical administration of an ophthalmic topical cream composition as described herein.

For example, an embodiment can provide a method for administering an ophthalmic medicine to a patient's eye, the method comprising applying an ophthalmic topical cream composition to an outer surface of an eyelid of the patient's eye, the topical cream comprising at least one zwitterion; and an active pharmaceutical ingredient (API) dispersed in the topical cream. The amounts and types of the various ingredients and the indications are described above with respect to the ophthalmic topical cream composition.

In various embodiments, the API can be pilocarpine and the method of treatment can be effective to alleviate the symptoms of dry eye. In another embodiment, the API can be pilocarpine and the method of treatment can be effective to treat presbyopia. In an embodiment, the API can comprise or can be travoprost and the method of treatment can be effective to treat intraocular pressure (IOP). In an embodiment, the API can comprise or can be physostigmine and the method of treatment can be effective to treat blepharitis. In an embodiment, the API can comprise or can be donepezil and the method of treatment can be effective to treat intraocular pressure (IOP).

In an embodiment, the method can comprise applying the ophthalmic topical cream composition to the outer surface of the eyelid of the patient's eye by applying with an applicator. For example, in an embodiment, the treatment can comprise squeezing ophthalmic topical cream composition from a container onto the applicator (such as a tube attached to the container) and then using the applicator to apply the ophthalmic topical cream composition to the outer surface of the eyelid of the patient's eye, e.g., by further squeezing the container to push the ophthalmic topical cream composition from the applicator onto the eyelid.

In various embodiments, the method of treatment can comprise applying the ophthalmic topical cream composition to the outer surface of the eyelid of the patient's eye at least once per day. For example, in an embodiment, the method can comprise applying the ophthalmic topical cream composition to the outer surface of the eyelid of the patient's eye up to four times per day, such as once per day, twice per day, three times per day or four times per day.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

### Example Embodiments

1. An ophthalmic topical cream composition, comprising:
a topical cream comprising at least one zwitterion; and
an active pharmaceutical ingredient (API) dispersed in the topical cream.

2. The ophthalmic topical cream composition of claim 1, wherein the zwitterion is present in an amount effective to reduce degradation of the API and enhance viscosity of the topical cream.

3. The ophthalmic topical cream composition of claim 1 or 2, wherein the at least one zwitterion has a concentration in the topical cream of about 5 mM to about 600 mM.

4. The ophthalmic topical cream composition of any one of claims 1 to 3, wherein the at least one zwitterion has a concentration in the topical cream of about 10 mM to about 500 mM.

5. The ophthalmic topical cream composition of any one of claims 1 to 4, wherein the topical cream has a viscosity of about 25,000 cPs to about 400,000 cPs.

6. The ophthalmic topical cream composition of any one of claims 1 to 5, wherein the topical cream has a viscosity of about 50,000 cPs to about 150,000 cPs.

7. The ophthalmic topical cream composition of any one of claims 1 to 6, wherein the at least one zwitterion comprises at least one of an amino acid, a peptide, a protein, a phosphatidylcholine, and a betaine.

8. The ophthalmic topical cream composition of any one of claims 1 to 7, wherein the topical cream is an oil-in-water or water-in-oil emulsion system.

9. The ophthalmic topical cream composition of claim 7 or 8, wherein the at least one amino acid comprises lysine, arginine, histidine, or a combination thereof.

10. The ophthalmic topical cream composition of claim 9, wherein the at least one amino acid comprises lysine and arginine.

11. The ophthalmic topical cream composition of claim 10, wherein the lysine and arginine in the topical cream each independently has a concentration of about 10 mM to about 500 mM.

12. The ophthalmic topical cream composition of claim 9, wherein the at least one amino acid comprises histidine and arginine.

13. The ophthalmic topical cream composition of claim 12, wherein the histidine and arginine in the topical cream each independently has a concentration of about 10 mM to about 500 mM.

14. The ophthalmic topical cream composition of claim 9, wherein the at least one amino acid comprises histidine and lysine.

15. The ophthalmic topical cream composition of claim 14, wherein the histidine and lysine in the topical cream each independently has a concentration of about 10 mM to about 500 mM.

16. The ophthalmic topical cream composition of claim 1, wherein the at least one zwitterion comprises a zwitterionic polymer or a polymeric zwitterion.

17. The ophthalmic topical cream composition of any one of claims 1 to 16, wherein the topical cream further comprises a crosslinked acrylic acid polymer.

18. The ophthalmic topical cream composition of any one of claims 1 to 16, wherein the topical cream comprises less than about 1% (w/w) of a crosslinked acrylic acid polymer.

19. The ophthalmic topical cream composition of any one of claims 1 to 18, wherein the topical cream has a pH of about 3.0 to about 7.8.

20. The ophthalmic topical cream composition of any one of claims 1 to 19, wherein the topical cream comprises about 0.002% to about 10% (w/w) of the API.

21. The ophthalmic topical cream composition of any one of claims 1 to 20, wherein the API comprises pilocarpine, atropine, travoprost, physostigmine, donepezil, timolol, loteprednol, brimonidine, and/or a salt of any of the foregoing.

22. The ophthalmic topical cream composition of any one of claims 1 to 21, wherein the topical cream comprises an amount of the API that is effective to alleviate the symptoms of dry eye, presbyopia, myopia, blepharitis, glaucoma, and/or edema.

23. A method of treating dry eye, presbyopia, glaucoma, and/or myopia, comprising administering an effective amount of the ophthalmic topical cream composition of any one of claims 1 to 22 to a subject in need thereof.

24. The method of claim 23, wherein the ophthalmic topical cream composition is applied to an outer surface of an eyelid of the subject's eye.

1. An ophthalmic topical cream composition, comprising:
a topical cream comprising at least one zwitterion; and
an active pharmaceutical ingredient (API) dispersed in the topical cream.

2. (canceled)

3. The ophthalmic topical cream composition of claim 1, wherein the at least one zwitterion has a concentration in the topical cream of about 5 mM to about 600 mM.

4. (canceled)

5. The ophthalmic topical cream composition of claim 1, wherein the topical cream has a viscosity of about 25,000 cPs to about 400,000 cPs.

6. (canceled)

7. The ophthalmic topical cream composition of claim 1, wherein the at least one zwitterion comprises at least one of an amino acid, a peptide, a protein, a phosphatidylcholine, and a betaine.

8. The ophthalmic topical cream composition of claim 1, wherein the topical cream is an oil-in-water or water-in-oil emulsion system.

9. The ophthalmic topical cream composition of claim 7, wherein the at least one amino acid comprises lysine, arginine, histidine, or a combination thereof.

10. The ophthalmic topical cream composition of claim 9, wherein the at least one amino acid comprises lysine and arginine.

11. The ophthalmic topical cream composition of claim 10, wherein the lysine and arginine in the topical cream each independently has a concentration of about 10 mM to about 500 mM.

12. The ophthalmic topical cream composition of claim 9, wherein the at least one amino acid comprises histidine and arginine.

13. The ophthalmic topical cream composition of claim 12, wherein the histidine and arginine in the topical cream each independently has a concentration of about 10 mM to about 500 mM.

14. The ophthalmic topical cream composition of claim 9, wherein the at least one amino acid comprises histidine and lysine.

15. The ophthalmic topical cream composition of claim 14, wherein the histidine and lysine in the topical cream each independently has a concentration of about 10 mM to about 500 mM.

16. The ophthalmic topical cream composition of claim 1, wherein the at least one zwitterion comprises a zwitterionic polymer or a polymeric zwitterion.

17. The ophthalmic topical cream composition of claim 1, wherein the topical cream further comprises a crosslinked acrylic acid polymer.

18. The ophthalmic topical cream composition of claim 1, wherein the topical cream comprises less than about 1% (w/w) of a crosslinked acrylic acid polymer.

19. The ophthalmic topical cream composition of claim 1, wherein the topical cream has a pH of about 3.0 to about 7.8.

20. The ophthalmic topical cream composition of claim 1, wherein the topical cream comprises about 0.002% to about 10% (w/w) of the API.

21. The ophthalmic topical cream composition of claim 1, wherein the API comprises pilocarpine, atropine, travoprost, physostigmine, donepezil, timolol, loteprednol, brimonidine, and/or a salt of any of the foregoing.

22. The ophthalmic topical cream composition of claim 1, wherein the topical cream comprises an amount of the API that is effective to alleviate the symptoms of dry eye, presbyopia, myopia, blepharitis, glaucoma, and/or edema.

23. A method of treating dry eye, presbyopia, glaucoma, and/or myopia, comprising administering an effective amount of the ophthalmic topical cream composition of claim 1 to a subject in need thereof.

24. (canceled)
